(19) United States
(12) Patent Application Publication
(10) Pub. No.: US 2025/0072718 A1
(43) Pub. Date: Mar. 6, 2025

(54) ENDOSCOPE COMPRISING A SUCTION VALVE

(71) Applicant: AMBU A/S, Ballerup (DK)

(72) Inventors: Alistair David MORTON, Copenhagen (DK); Anders KLÜVER, Ballerup (DK); Thomas Bachgaard JENSEN, Værløse (DK)

(73) Assignee: AMBU A/S, Ballerup (DK)

(21) Appl. No.: 18/824,143

(22) Filed: Sep. 4, 2024

(30) Foreign Application Priority Data
Sep. 5, 2023 (EP) 23195488.4
Sep. 5, 2023 (EP) 23195491.8
Feb. 28, 2024 (EP) 24160240.8
Aug. 29, 2024 (EP) 24197365.0

(51) Int. Cl. A61B 1/00; A61B 1/005; A61B 1/012

(52) U.S. Cl. CPC A61B 1/00068; A61B 1/00043; A61B 1/00128; A61B 1/005; A61B 1/012

(57) ABSTRACT

An endoscope including a handle or interface including a suction valve and a housing made from a first half-shell and a second half-shell. The suction valve is mounted between the first half-shell and the second half-shell. The first half-shell includes first and second fixing portions to receive first and second fixing portions of the suction valve. The second half-shell comprises third and fourth fixing portions configured to receive the second portion of the suction valve. The second and fourth fixing portions have a different geometry, the second fixing portion is configured to suppress at least one degree of freedom of the suction valve, and the fourth inner fixing portion is configured to suppress a remaining degree or remaining degrees of freedom of the suction valve, for fixing the suction valve in its position and orientation in the housing.

### CROSS-REFERENCE TO RELATED APPLICATIONS

This application claims priority from and the benefit of European Patent Application No. 23 195 488.4 and European Patent Application No. 23 195 491.8, both filed Sep. 5, 2023, European Patent Application No. 24 160 240.8, filed Feb. 28, 2024, and European Patent Application No. 24 197 365.0, filed Aug. 29, 2024; the disclosures of said applications are incorporated by reference herein in their entirety.

### TECHNICAL FIELD

The present disclosure relates to an endoscope comprising a suction valve mounted on the endoscope's handle. The present disclosure further relates to a method of assembling the suction valve of the endoscope.

### BACKGROUND

Endoscopes, including specialized instruments such as bronchoscopes, arthroscopes, colonoscopes, laparoscopes, gastroscopes, and duodenoscopes, are well-known and are typically used for visual examination and diagnosis of hollow organs and body cavities, as well as to assist in surgery, e.g. for a targeted tissue sampling. Both reusable and disposable endoscopes are known. Single-use endoscopes mitigate or eliminate the risk for cross-contamination and improve availability of such instruments where and when they are needed in addition to eliminating the time and cost involved to clean re-usable endoscopes. To reduced cost, single-use endoscopes are often made with a relatively high polymer content and are assembled using adhesives.

When examining an object such as a body cavity or hollow organ with an endoscope, it is desirable to have a clear and good view or visibility of the examined object. However, the visibility of such an object is often affected by undesirable fluid or mucus, which may obstruct visibility of the object to be examined. Furthermore, it may be desirable to remove tissue parts or particles during the surgery, for example to extract a sample for further analysis, or to provide better visibility. It is thus desirable to remove such undesirable fluid or mucus, as well as such tissue parts or particles, using a suction device, such as a vacuum pump. A surgeon can start and interrupt suction by actuating a vacuum pump providing the suction. However, actuation of the pump to control suction results in considerable delays caused by starting and stopping of the pump's motor and potentially longer fluid flows since the pump is not located adjacent the handle.

To overcome these disadvantages, suction valves comprising housings and pistons movable within the housings can switch the vacuum on and off without shutting off the vacuum pump. However, with these suction valves fluid, mucus and tissue sucked-off by the suction device is sharply deflected several times within the suction valve, which considerably reduces the volume flow, increases the load on the vacuum pump, and reduces the pumping capacity and suction performance. Furthermore, narrowing of the valve increases the risk of clogging, which leads to interruptions in the medical procedure, potentially endangering a patient's safety. Additionally, known suction valves are complex, with a large number of parts, which makes the valves expensive to manufacture, which is of considerable disadvantage, especially for the use of such a suction valve with a single-use endoscope. Especially in single-use endoscopes, the suction valve is usually glued into the housing of the handle. Such gluing involves considerable assembly work, which makes the endoscope more complex to manufacture and thus more expensive.

Furthermore, studies have shown that the carbon footprint of maintaining and cleaning re-usable endoscopes may be equal to or greater than the carbon footprint of single-use endoscopes. However, it is important to continue reducing the carbon footprint of single-use devices. Designing single-use devices with lower carbon footprint materials comes with drawbacks, however, since such materials may have different characteristics in a range of variables including temperature, adhesion, shrinking, warpage, rigidity, and strength.

### BRIEF DESCRIPTION OF THE DISCLOSURE

The object of the present disclosure is therefore to overcome or at least reduce the disadvantages of the related art. More specifically, it is the purpose of the present disclosure to provide an endoscope having a suction valve, wherein the endoscope is formed from components that are simple and inexpensive to manufacture, and wherein the endoscope is easy to assemble.

It is also an object of the present disclosure to provide a single-use endoscope with even lower carbon footprint than prior single-use endoscopes. The carbon footprint may be reduced by selecting lower carbon footprint materials and reducing the use of adhesives to bond plastic components. Synergistically, by configuring components in novel ways the reduction of the carbon footprint may result in lower material and assembly costs.

In one aspect, the present disclosure provides an endoscope comprising a suction valve mounted on the endoscope's handle. In a second aspect, the present disclosure provides a method of assembling the suction valve of the endoscope. In a third aspect, the present disclosure provides a visualization system including the endoscope according to the first aspect.

In one embodiment according to the first aspect, the endoscope that comprises a proximal handle or interface, an insertion cord extending from the handle or interface and configured to be inserted into a patient's body cavity, and a working channel configured to let fluid and mucus sucked from the patient's body cavity flow therethrough. The handle or interface comprises a housing and a suction valve configured to control a suction through the working channel and having a valve closed state and a valve open state.

The housing comprises a first half-shell and a second half-shell. The suction valve comprises a valve body having an inlet connected to the working channel, an outlet provided and configured to be connected to a suction device and a stem movable axially in the valve body between at least a first position, in which the suction valve is in the valve closed state, and a second position, in which the suction valve is in the valve open state. The suction valve is mounted or mountable between the first half-shell and the second half-shell. The first half-shell comprises a first, outer fixing portion and a second, inner fixing portion. The first, outer fixing portion is configured to receive a first portion of the suction valve and the second, inner fixing portion is configured to receive a second portion of the suction valve.

Preferably, the second half-shell comprises a third, outer fixing portion and a fourth, inner fixing portion, wherein the third, outer fixing portion is configured to receive the first portion of the suction valve, and wherein the fourth, inner fixing portion is configured to receive the second portion of the suction valve.

Preferably, the second, inner fixing portion of the first half shell and the fourth inner fixing portion of the second half shell have a different geometry or a different shape.

Especially preferred, the second, inner fixing portion is configured to suppress or constrain at least one degree of freedom of the suction valve, in particular of the valve body of the suction valve, and the fourth, inner fixing portion is configured to suppress or constrain a remaining degree or remaining degrees of freedom of the suction valve, in particular of the valve body of the suction valve, for fixing the suction valve in its position and orientation in the housing of the handle or interface.

It is to be understood, that a body like the suction valve has six degrees of freedom in space, three rotational degrees of freedom and three translational degrees of freedom.

“Suppressing” a degree of freedom is to be understood as preventing an arbitrary translational or rotational movement of the suction valve in or relative to the housing of the endoscope at least in one direction along an axis of a Cartesian coordinate system or at least around an axis of the Cartesian coordinate system. “Suppressing” can be understood as “constraining”.

It is to be understood, that the degrees of freedom of the suction valve refer to the suction valve as a whole and in particular to the valve body, however not to the stem. In other words, if degrees of freedom or movements of the suction valve are suppressed, this means a fixation of the suction valve and in particular of the valve body relative to the housing of the endoscope but does not mean that e.g. the stem may be prevented from moving.

The handle may be substituted by the interface. Alternatively, it is conceivable that the interface is formed on the handle. The handle may contain essential control elements, such as a lever for controlling bending of the endoscope for the endoscope and ports for accessories such as a video processing apparatus or the suction device, for example in the form of a vacuum pump. The handle is preferably designed in such a way that it can be ergonomically gripped and operated by the user or operator or surgeon with just one hand. This means that all important operating elements on the handle are preferably arranged in such a way that the user can operate them with one hand without having to change a position of his or her hand. The handle may comprise a shell. Preferably, the shell is made from a plastic material. Furthermore, the shell preferably comprises two half-shells, a first half-shell and a second half-shell which are connectable with each other in a separation plane or dividing plane or split plane. In case of a provision of a proximal endoscope interface, said endoscope interface may be prepared or provided to be connectable to e.g. a robotic arm.

Handles, and other components of single-use endoscopes, are typically manufactured from acrylonitrile butadiene styrene (ABS). ABS is an amorphous polymer suitable for applications that require high strength, stiffness, and heat resistance, while polypropylene (PP), a semi-crystalline polymer, is more suitable for applications that require high flexibility. For example, the elongation at break of ABS is about 30% while the elongation at break of PP may be about 100%. Further, ABS requires more heat to maintain the polymer at a high level while feeding the material to an injection molding machine. Otherwise, the material will cool quickly which can result in structural flaws in the final product. And ABS, due to being amorphous, does not lend itself well to on-the-fly processing changes during manufacturing, which changes result in more scrapped material relative to PP. In sum, an endoscope according to the present disclosure reduces carbon footprint by using PP instead of ABS and provides novel features especially configured to solve the problems caused by such substitution, including lower rigidity and lower adhesion characteristics. The carbon footprint can be reduced further by using bio-PP instead of carbon based PP.

The endoscope further comprises the insertion cord, preferably having a distal tip unit formed at the distal end of the insertion cord. The distal tip unit may comprise an image capturing means like a camera and a light emitting device like a LED.

Furthermore, the endoscope comprises the working channel. The working channel is provided and configured to transport fluid, mucus and tissue parts or particles from the patient's body cavity. Furthermore, the working channel may be configured to guide a surgical tool or instrument through it to a surgical location in the patient's body. For this purpose, the working channel may have a Y-connector in the handle that allows insertion of the surgical tool or instrument into the working channel.

The endoscope further comprises the suction valve arranged in the handle or interface.

The suction valve comprises the valve body, which may also be designated as a valve housing. The valve body may be formed cylindrically at least in portions and may thus be described as including a valve cylinder that extends in an axial direction of the suction valve.

The inlet and the outlet of the suction valve are preferably formed as integral parts or portions of the valve body. The inlet and the outlet may have substantially a geometry of a pipe section, preferably of a pipe section with an annular cross-section.

The inlet and outlet may extend away from an outer cylindrical surface of the valve cylinder of the valve body.

The inlet is provided and configured to be connected to the working channel. The inlet may be connected directly to the working channel. Alternatively, the inlet may be connected to the working channel via a suction channel, especially preferably to the Y-connector of the working channel. The suction channel may e.g. be configured as a separate hose or tube.

The outlet is provided and configured to be connected to the suction device. The outlet may be connected directly to the suction device. Alternatively, a hose may be formed between the outlet and the suction device. Alternatively or additionally, at least one coupling element and/or at least one deflector element and/or at least one pipe element may be formed between the outlet and the suction device.

The suction valve further comprises the stem. The stem is arranged in the valve body in such a way that the stem is able to move in the axial direction of the suction valve in the valve body, especially in the valve cylinder of the valve body. The axial movement, at least in one direction, can be caused by pushing a button. The button may be a button portion which is preferably formed integrally with preferably a remainder of the stem at an end portion of the stem. Alternatively, the button may be a button element attached to the stem, in particular to the end portion of the stem. The button portion or element may protrude axially from the valve body, so that a user can press the button portion or element in order to move the stem axially in the valve body. The stem can be moved in the valve body in such a way that it shifts the suction valve at least between the valve closed state of the suction valve, in other words in its first position, and the valve open state of the suction valve, in other words in its second position. In other words, the stem may be configured to seal between an inlet opening and an outlet opening in the suction valve closed state, and the stem may be configured to enable a fluid flow between the inlet opening and the outlet opening of the valve body in the suction valve open state. In still other words, the suction valve may be configured to manually control the suction through the working channel, in particular by the user pressing the button towards the housing of the endoscope. In this way, the user can easily control the suction valve and can switch between the valve closed state and the valve open state.

The suction valve is mounted between the first half-shell and the second half-shell. In other words, the suction valve is arranged in the dividing plane of the handle and is fixed to the first half-shell and/or the second half-shell.

The suction valve may be locked in the first half-shell and/or the second half-shell.

The first half-shell comprises the first fixing portion and the second fixing portion. The first fixing portion is positioned further outwardly than the second fixing portion. In other words, the first fixing portion is positioned closer to a circumferential or peripheral edge of the shell, which is positioned in the dividing plane, than the second fixing portion.

The first fixing portion is provided and configured to receive the first portion of the suction valve. “Receiving” means that the first portion is held by the first fixing portion, for example in a form fitting manner. Alternatively or additionally, a frictional connection such as press fit may be provided.

The second fixing portion is provided and configured to receive the second portion of the suction valve. “Receiving” means that the second portion is held by the second fixing portion, for example in a form fitting manner. Alternatively or additionally, a frictional connection may be provided.

The first portion of the suction valve is different from the second portion of the suction valve. In other words, the first portion is spaced from the second portion.

Preferably, the first portion and the second portion of the suction valve may be formed of different materials.

Further preferably, the first portion and the second portion may have substantially different geometric shapes. For example, the first portion may have a round geometry and the second portion may have an angular geometry.

Such a configuration of the endoscope allows the suction valve to be positioned and fixed in the handle easily and without the use of tools. The formation of two different mounting points between the suction valve and the first half-shell ensures that the suction valve is firmly fixed in the handle without twisting. Furthermore, such a configuration can prevent incorrect assembly, for example by the poka yoke principle.

The suction valve, in particular the parts thereof, may be made of a plastic material or polymer. Especially preferred, the suction valve, in particular parts thereof, preferably the valve body and the stem, may be manufactured in an injection molding process, i.e. may be injection molded parts. The suction valve may comprise at least 70% polypropylene polymer, or may consist essentially of polypropylene polymer.

The second half-shell may comprise a third, outer fixing portion and a fourth, inner fixing portion. The third, outer fixing portion may be configured to receive the first portion of the suction valve, and the fourth, inner fixing portion may be configured to receive the second portion of the suction valve.

The third fixing portion may be provided and configured to receive the first portion of the suction valve. “Receiving” means that the first portion is held by the third fixing portion, for example in a form fitting manner. Alternatively or additionally, a frictional connection may be provided. The third fixing portion may be configured to suppress a degree of freedom of the suction valve, in particular at least partially or in one direction.

The fourth fixing portion may be provided and configured to receive the second portion of the suction valve. “Receiving” means that the second portion is held by the fourth fixing portion, for example in a form fitting manner. Alternatively or additionally, a frictional connection may be provided. The fourth fixing portion may be configured to suppress a degree of freedom of the suction valve, in particular at least partially or in one direction.

Since the first half-shell and the second half-shell are mounted to each other to form the handle, it is advantageous if the third fixing portion and the fourth fixing portion, which also secure the suction valve, are formed on the second half-shell. In this way, the mounting of the suction valve can be further improved and a more rigid connection between the first half-shell, the second half-shell and the suction valve can be achieved.

In a variation of the present embodiment, the first outer fixing portion of the first half-shell and the third outer fixing portion of the second half-shell may be formed identically or symmetrically to each other. In other words, the third fixing portion of the second half-shell may correspond to the first fixing portion of the first half-shell, and/or the third fixing portion of the second half-shell may correspond to the first fixing portion of the first half-shell mirrored on the split plane or dividing plane of the handle. Such a matched configuration of the first fixing portion and the third fixing portion ensures uniform force transmission between the suction valve, the first half-shell and the second half-shell. This is particularly advantageous because the first fixing portion and the third fixing portion are each the outer fixing portions, which are close to a force application point of the suction valve.

In another variation of the present embodiment, the first, outer fixing portion of the first half-shell and the third, outer fixing portion of the second half-shell may preferably substantially completely encircle the first portion of the suction valve in a circumferential direction.

The first fixing portion and the third fixing portion may each have a semi-circular shape which, in the assembled state, that is, in the state when the first half-shell is assembled to the second half-shell and thus the housing or handle shell is formed, form a circular shape which completely encloses the first portion of the suction valve, which may be circular in shape.

The first fixing portion and the third fixing portion may form a collar shape that encloses the first portion of the suction valve.

Such a configuration of the first fixing portion and the third fixing portion can, in addition to further improving the force transmission, implement a seal between the suction valve and the handle. In particular, the sealing can be implemented without additional sealing elements, which further reduces the cost of the endoscope and makes the endoscope easier and cheaper to assemble.

In another variation of the present embodiment, the first fixing portion and/or the third fixing portion may be formed as a portion of the handle shell, preferably of the half shell, having a groove or a recess.

The second, inner fixing portion of the first half-shell and the fourth inner fixing portion of the second half-shell may be formed differently.

The second fixing portion and the fourth fixing portion may have different geometries of different shapes or, they may differ more or further than just being mirrored. For example, the second fixing portion may have a cylindrical shape or comprise a cylinder and the fourth fixing portion then may not have that cylindrical shape or comprise such a cylinder but e.g. have a rectangular shape. The second fixing portion may comprise shapes or shaped portions, that the fourth fixing portion does not comprise.

Preferably, the mounting in the second fixing portion may differ from the mounting in the fourth fixing portion. Alternatively or additionally, a clearance of the second fixing portion may differ from a clearance of the fourth fixing portion.

Different fixations in the second fixing portion and the fourth fixing portion can facilitate an assembly process of the handle. Furthermore, excessive tensioning of the suction valve between the second fixing portion of the first half-shell and the fourth fixing portion of the second half-shell can be prevented by the different fixations.

In another aspect, the second, inner fixing portion of the first half-shell and the fourth inner fixing portion of the second half-shell may be configured as tower geometries protruding from outer shell portions or shell skins of the half-shells into an interior space of the handle or interface.

The tower geometries make it possible to hold the suction valve in the center of the handle without having to make the corresponding portion on the suction valve too long, which could reduce the stability of the portion.

As described above, the second, inner fixing portion may be configured to suppress at least one degree of freedom of the suction valve and the fourth, inner fixing portion may be configured to suppress a remaining degree or remaining degrees of freedom of the suction valve, so that the suction valve is fixed in its position and orientation in the housing of the handle or interface. It is to be understood that this preferably means that it is prevented that the suction valve may change its position or orientation in the endoscope housing. It is to be understood that the second, inner fixing portion may be configured to suppress at least one degree of freedom of the suction valve and the fourth, inner fixing portion is configured to suppress a remaining degree or remaining degrees of freedom of the suction valve, but that does not necessarily mean that the second, inner fixing portion and the fourth, inner fixing portion are together configured to suppress all degrees of freedom of the suction valve by themselves, i.e. alone, this is only preferred. As described above, the first and/or third, outer fixing portions may also suppress a degree of freedom of the suction valve, in particular at least partially or in one direction, therefore it is not necessary that this degree of freedom is then also suppressed by the second, inner fixing portion or the fourth, inner fixing portion. In other words, the fourth, inner fixing portion may be configured to suppress a degree or degrees of freedom that are not suppressed by the first, second or third fixing portions.

Preferably, the second, inner fixing portion and the fourth, inner fixing portion are configured to suppress together all degrees of freedom of the suction valve, in particular three positional and three rotational degrees of freedom. Preferably the second, inner fixing portion and the fourth, inner fixing portion are configured to fixate together the suction valve in position and orientation.

Preferably, the second, inner fixing portion is configured to suppress more degrees of freedom of the suction valve than the fourth, inner fixing portion.

Preferably, the second, inner fixing portion is configured to suppress (due to its shape) translational movements except in a direction towards the second half-shell and/or to the fourth fixing portion, when the suction valve is inserted, i.e. in the assembled state of the endoscope. Preferably, the fourth, inner fixing portion (of the second half-shell) is configured to prevent/suppress this movement.

Preferably, the second, inner fixing portion is configured to only allow a translational movement of the suction valve in direction of or to the second half-shell, when the suction valve is inserted, i.e. in the assembled state of the endoscope. In other words, preferably the second, inner fixing portion is configured to suppress by its shape arbitrary rotations and all translations but one translational movement. It is to be understood that the second, inner fixing portion may nevertheless provide some small rotational looseness by its shape making it easier to insert a corresponding portion of the suction valve.

Preferably, the fourth, inner fixing portion of the second half-shell is configured to prevent or suppress this remaining one translational movement. This makes an assembly easier and allows critical tolerances for fixation of the suction valve to be limited to the first half-shell.

Preferably, the second, inner fixing portion of the first half-shell has a receptacle configured to hold or receive a portion of the suction valve, such as a fixing projection of the suction valve.

In the following, the receptacle of the second, inner fixing portion is described:

Preferably, the receptacle of the second, inner fixing portion has substantially a polygonal shape or cross-section. More preferably, it has a substantially rectangular cross-section and is therefore slot-shaped.

Preferably, the rectangular cross-section tapers slightly. A rectangular cross-section is to be understood as a cross-section, which can hold or receive another body of rectangular shape. It may comprise a split slot and/or be made of or comprise two separate portions.

Preferably, the second, inner fixing portion is made of or comprises two separate portions or brackets spaced from each other.

Preferably, these two portions are both formed as first tower geometries, protruding from the first half-shell in direction towards the centre of the housing and/or the second half-shell.

Preferably, these two portions are mirrored portions. Preferably, one of these portions may result by mirroring the other one at a mirror plane between them.

Preferably, the two portions are U-shaped brackets opposed to each other forming the slot-shaped receptacle. U-shaped is to be understood as being able to cover something inside from three sides, at least partially. Preferably, the two U-shaped brackets form a split slot in between them. Such a split slot can add flexibility and save material.

The split slot is preferably formed in between the first tower geometries or in other words in between two portions of the second, inner fixing portion. A first tower geometry may be understood as a geometry, which extends preferably perpendicularly away from an outer shell portion of the first half-shell in the direction of the interior of the handle. Preferably, the extension towards the interior is greater than an extension parallel to the outer shell portion or shell skin.

Preferably, the receptacle may taper slightly.

More preferably, the receptacle is reduced linearly and/or continuously going further inside, in direction from the center of the housing to an outer shell portion of the first half-shell. In other words, the receptacle may have an angled interface, e.g. a drafting angle.

Preferably, the receptacle has at least one angled interface or wall or inner surface portion also serving as an inlet chamfer.

Therefore, the suction valve or a portion of the suction valve may be press-fitted inside. In other words, the receptacle may be configured for a press-fit with a corresponding portion of the suction valve.

Preferably, the receptacle tapers slightly in two directions or dimensions, in particular in width and height, wherein the third axis going inside the receptacle(s) may be described as length. Preferably, the cross-section of the receptacle is rectangular, having a width and a height. Especially in this case, the two sides opposite to each other, respectively, may get closer to each other further inside the receptacle. In other words, the width and height of the receptacle may reduce.

Additionally or alternatively, the receptacle may be of constant width and/or height and/or configured for a form-fit with a corresponding portion of the suction valve. In other words, the taper of the receptacle of the second, inner fixing portion may be small so that the second, inner fixing portion of the first half-shell may be defined as comprising a slot of essentially constant width, despite of the small taper. It may also be provided that the slot is exactly of constant width, i.e. without taper.

Preferably, the fourth, inner fixing portion of the second half-shell may comprise a receptacle configured to hold or receive a portion of the suction valve, such as a fixing projection of the suction valve.

In the following, the receptacle of the fourth, inner fixing portion is described:

Preferably, the receptacle of the fourth, inner fixing portion is V-shaped and thereby configured to guide the suction valve or a portion of the suction valve into it, in particular during assembly.

Preferably, the V-shaped receptacle guides, during assembly, the suction valve into it in a, preferably the only, direction of movement of the suction valve that is not suppressed by the shape of the second, inner fixing portion. In other words, preferably the V-shaped receptacle guides the suction valve during assembly into it in a direction, wherein the second, inner fixing portion does not suppress movement of the suction valve in that direction by its shape. This makes the assembly much easier.

It is to be understood, that the term V-shaped does not mean that it necessarily has completely the shape of a “V”, but substantially and therefore guiding the suction valve inside this V-shaped receptacle and making the assembly easier. In other words, V-shaped is not to be understood as purely geometrical but more as functional.

Preferably, the V-shaped receptacle of the fourth, inner fixing portion may comprise a force-taking surface portion as an inner surface of the receptacle that is provided to be in contact with the suction valve. Preferably, this force-taking surface portion is planar or flat and preferably perpendicular to a direction of the axis of the stem and/or a direction into which the button may be pressed and/or located in the V-shaped receptacle on a side opposite to the stem and/or button or on a side opposite to the direction in which the button is configured to be pressed. Thereby, when the button is pressed, this force-taking surface portion may take the pressing force from the suction valve and provide stability. Further, it may prevent a rotational and/or a translational movement of the suction valve due to the pressing force.

The force-taking surface portion may be perpendicular to a direction in which the stem is axially movable and/or the button may be pushed, and/or may be located on an inner side of the V-shaped receptacle opposite to the stem and/or button.

The, preferably flat, force-taking surface portion may be perpendicular to a direction in which the button of the suction valve is pushable to move the stem, so that if or when the button is pushed, a movement of the suction valve caused by the pushing of the button in that direction relative to the housing is suppressed by the flat force-taking surface portion.

Preferably, the V-shaped receptacle may comprise a planar or flat end surface portion as end or landing or docking surface of the receptacle. Preferably, this is an inner portion being the furthest inside the V-shaped receptacle, thereby flattening the V-shaped receptacle so that it is not pointy. Preferably, this end surface portion is provided and/or configured so that the suction valve or a portion of the suction valve may be adjacent to it or abut it flatly, when the endoscope is assembled.

The V-shaped receptacle of the fourth, inner fixing portion may preferably be formed on a second tower geometry. A second tower geometry may be understood to be a geometry, which extends substantially perpendicularly away from an outer shell portion or shell skin of the second half-shell in the direction of the interior of the handle. Preferably, the extension towards the interior is greater than an extension parallel to the outer shell portion or shell skin.

Preferably, the fourth, inner fixing portion is made of or comprises two separate portions or brackets spaced from each other.

Preferably, these two brackets are both second tower geometries protruding from the second half-shell in direction towards the centre of the housing and/or the first half-shell.

Preferably, both these two brackets are shaped identical and/or facing each other.

Such a configuration of the second fixing portion and the fourth fixing portion can further simplify the assembly of the handle. The second substantially slot-shaped fixing portion in the first half-shell can firmly position the suction valve, and the V-shape of the fourth fixing portion can significantly simplify the process of introducing the portion of the suction valve into the fourth fixing portion, which is particularly advantageous when assembling the first half-shell with the second half-shell, where a view of the fourth fixing portion is obscured by the second half-shell.

In a further variation of the present embodiment, the first portion of the suction valve may be the valve spring.

The first fixing portion of the first half-shell and/or the third fixing portion of the second half-shell may contact and hold the valve spring of the suction valve, preferably circumferentially.

The valve spring may preferably be formed by an elastic material, in particular an elastomer.

By fixing the suction valve via the valve spring, a reliable sealing between the handle and the suction valve can be achieved. This sealing can be further increased if the valve spring is made of an elastic material, in particular an elastomer. In addition, the formation of further fastening geometries on the suction valve can be omitted, which reduces the number of components or the complexity of the components and thus the costs and assembly effort for the suction valve and the endoscope.

In a further variation of the present embodiment, the second portion of the suction valve may be at least one projection formed integrally with the valve body.

The second portion may be an integral section of the valve body, which is injection molded together with the valve body. The second portion may include at least one projection, preferably two projections.

In a yet further variation of the present embodiment, the second portion may comprise a first projection formed integrally with the valve body and a second projection formed integrally with the valve body, said first projection and said second projection may project in opposite directions from said valve body, in particular from the valve cylinder of the valve body.

The second portion may comprise the two wing-shaped projections formed radially opposite each other on the valve body. The first projection and the second projection may be symmetrical or identical. Alternatively, it is conceivable to form the first projection and the second projection differently, for example to implement the poka yoke principle. The first projection and the second projection may differ in width, length, thickness and/or cross-sectional geometry.

In a further variation of the present embodiment, the second portion of the suction valve may comprise a stop, which defines a maximum penetration depth of the second portion.

The first projection and/or the second projection may comprise a stop. The stop may be in the form of a reinforcing rib or in the form of an annular rib or in the form of shoulders formed on the projection or the like.

Such a stop can simplify the assembly of the handle, reducing assembly time and thus assembly costs, since an assembler does not have to align the projection manually but simply pushes it in until the stop prevents further insertion.

In another aspect, the first half-shell may comprise retention hooks, which are formed directly adjacent to at least the first, outer fixing portion to close the housing.

Latching geometries in the form of retention hooks may be formed between the first half-shell and the second half-shell to allow the handle to be closed or assembled without tools. In particular, the latching geometries may be formed directly adjacent to the first fixing portion to increase a clamping force on the first fixing portion and/or the third fixing portion to improve sealing and fixation of the suction valve in the handle.

Due to the integral design of the stem, the number of components of the suction valve can be further reduced, which further reduces the assembly effort and thus the assembly costs of the suction valve. Fewer components also reduces the amount of adhesive that is used to bond components of the endoscope to each other, further reducing the carbon footprint.

In another aspect, the valve body and/or the stem are preferably made from a thermoplastic polymer, e.g. polypropylene (PP) especially biodegradable polypropylene, polystyrene (PS), polycarbonate (PC), acrylonitrile butadiene styrene (ABS), polyoxymethylene (POM), etc. However, also other polymer materials or other materials are conceivable. However, polypropylene is preferred because it reduces the carbon footprint relative to many other polymers, such as PS, PC and ABS.

Bio-based PP is understood to originate from biomass, which is renewable, e.g., within a time span of up to 10 years or up to 50 years. This is opposed to so-called synthetic PP which is made from a fossil-based feedstock, such as crude oil, which is not renewable, at least not when considering a time span of up to e.g., a thousand years. One way to establish whether the PP is bio-based or based on a fossil feedstock, is by a carbon 14 analysis. A carbon 14 analysis can also provide an estimate of the relationship between the content of bio-based PP and the content of fossil-based PP. Bio-based polypropylene may be manufactured from natural materials such as lignocellulosic biomass (starch and cellulose), fatty acids, and organic waste, e.g., food waste or waste products from farming or forestry. Also, corn, sugar cane, vegetable oil, and some other types of biomass may be applied. One practical way to add bio-based PP as a fraction of the handle shell material is to apply the so-called bio-attributed concept, where the bio-based fraction of the PP is calculated from the mass balance approach (or model). This means that the fraction of bio-based PP in a component may not be known, but the average fraction over some time, or for a certain number of said components, is well known. The supplier of the PP raw material to the molding, will comply with the relevant standards (such as the series of ISO 16620 standards: ISO 16620-1:2015, ISO 16620-2:2019, ISO 16620-3:2015, ISO 16620-4:2016, ISO 16620-5:2017) related to the mass balance model. Applying this approach means that in order to know the bio-based fraction of PP in a specific component, or a specific batch of component, an analysis, such as an analysis of the carbon-14 content, may be necessary. The rest of the PP may come from fossil resources. Some of the PP could also be based on recycled PP.

Some changes in the design of the endoscope have been found to simplify the manufacturing of the endoscope and have enabled a shift from previous used polymers, such as ABS, into the use of PP, including bio-based PP. Recycled PP may comprise a combination of bio-based PP and fossil-based PP. As more and more bio-based PP is applied, the fraction of bio-based PP in recycled PP will increase. The content of bio-based PP does not influence the use of additives which may be added to the PP in order to provide specific, e.g., mechanical, characteristics to the endoscope material. The PP of the components may comprise at least 20% bio-based PP, alternatively, at least 40% bio-based polypropylene. The fraction of bio-based PP may also be at least 60%, or at least 80%. A higher fraction of bio-based PP means that the carbon dioxide emissions related to the endoscope during their life cycle will be lower. Some endoscope components, such as the valve and handle housing shells, may consist essentially of PP. Processing additives may be added as is known in the art. Alternatively, the components may comprise at least 60% PP, preferably more than 70% PP, of which at least 20% is bio-based PP. Fibers, such as natural fibers, may be added to modify physical properties, such as strength.

In a further variation of the present embodiment, the present disclosure further relates to an endoscope that comprises a proximal handle or interface, an insertion cord extending from the handle or interface and configured to be inserted into a patient's body cavity and a working channel configured to let fluid and mucus sucked from the patient's body cavity flow therethrough. The handle or interface comprises a housing and a suction valve configured to control a suction through the working channel and having a valve closed state and a valve open state. The housing comprises a first half-shell and a second half-shell The suction valve comprises a valve body having an inlet connected to the working channel, an outlet provided and configured to be connected to a suction device and a stem movable axially in the valve body between at least a first position, in which the suction valve is in the valve closed state, and a second position, in which the suction valve is in the valve open state. The suction valve is mounted between the first half-shell and the second half-shell. The first half-shell comprises a first, outer fixing portion and a second, inner fixing portion. The first, outer fixing portion is configured to receive a first portion of the suction valve and the second, inner fixing portion is configured to receive a second portion of the suction valve. The second half-shell comprises a third, outer fixing portion and a fourth, inner fixing portion. The third, outer fixing portion is configured to receive the first portion of the suction valve, and the fourth, inner fixing portion is configured to receive the second portion of the suction valve. The second, inner fixing portion of the first half-shell and the fourth inner fixing portion of the second half-shell have a different geometry. The second, inner fixing portion of the first half-shell comprises a slot, preferably of constant width, and the fourth inner fixing portion of the second half-shell comprises a (n essentially) V-shaped slot.

The object of the present disclosure is further solved by a method of assembling an endoscope as described above, comprising: sliding a valve spring onto a valve body of a suction valve and fixing or latching the valve spring to a stem of the suction valve; positioning the suction valve in a first half-shell of a housing of an handle or interface by inserting a second portion of the suction valve in a second, inner fixing portion of the first half-shell of the housing; inserting a first portion of the suction valve in a first, outer fixing portion of the first half-shell; positioning a second half-shell of the housing on the first half-shell of the housing; inserting the second portion of the suction valve in the fourth, inner fixing portion of the second half-shell of the housing; inserting the first portion of the suction valve in a third, outer fixing portion of the second half-shell; and closing the half-shells.

In other words, the object of the present disclosure is also solved by the method of assembling or mounting the endoscope according to any of the above aspects.

In a first step, the valve spring is fitted onto the valve body so that the valve body and valve spring are connected to each other. The connection between the valve spring and the valve body can be a force-fit and/or a form-fit connection.

In a second step, the suction valve is positioned in the first half-shell, with a first projection of the second portion of the valve body being inserted, preferably as far as the stop allows, into the second fixing portion.

In a third step, the first portion of the suction valve, which is preferably the valve spring, is inserted into the first fixing portion of the first half-shell, which preferably comprises a groove.

In a fourth step, the two half-shells are positioned relative to each other so that their openings are facing each other.

In a fifth step, the two half-shells are joined together, with the second projection of the second portion of the suction valve being inserted into the fourth fixing portion. The fourth fixing portion preferably includes the V-shape to facilitate insertion.

In a sixth step, the first portion of the suction valve, which is preferably the valve spring, is inserted into the third fixing portion of the second half-shell, which preferably comprises a groove.

In a seventh step, the first half-shell and the second half-shell are closed to form the handle of the endoscope. Preferably, latching hooks formed between the first half-shell and the second half-shell are engaged with each other. In other words, the first half-shell and the second half-shell are attached and/or assembled to each other.

The present disclosure further relates to a system comprising the endoscope according to one of the above aspects and a video processing apparatus (VPA) couplable to the endoscope and capable of outputting a live image recorded by an image sensor of the endoscope on a display. The video processing apparatus may comprise a built-in display and/or a detachable display and/or may be couplable to an external display. The video processing apparatus may be a video processing apparatus showing an image or a video captured by an image capturing means arranged at the distal tip unit.

Furthermore, the present disclosure relates to a system, that may also be claimed independently, comprising the endoscope described above wherein the system further comprises a suction device that is connectable directly or indirectly, for example through a hose, to the outlet of the valve body of the suction valve.

The suction device may be a vacuum pump or the like. There are also embodiments imaginable, in which the suction device is directly coupled to the endoscope or is part of the endoscope.

### BRIEF DESCRIPTION OF THE DRAWINGS

The disclosure is explained in more detail below using preferred embodiments and referring to the accompanying figures.

FIG. 1 shows a side view of an endoscope according to the present disclosure.

FIG. 2 shows a first half-shell of the handle or interface having a suction valve.

FIG. 3 shows the suction valve mounted in the first half-shell in an enlarged view.

FIG. 4 shows the first half-shell in a perspective view.

FIG. 5 shows a first enlarged view of the first half-shell.

FIG. 6 shows a second enlarged view of the first half-shell.

FIG. 7 shows a first enlarged view of a second half-shell.

FIG. 8 shows a second enlarged view of the second half-shell.

FIG. 9 shows a sectional view of the first half-shell and the second half-shell mounted to each other.

FIG. 10 shows a schematic view of the suction valve mounted in the handle.

FIG. 11 shows a sectional view of the suction valve mounted in the handle.

FIG. 12 shows a perspective view of the suction valve mounted in the first half-shell.

FIG. 13 shows a sectional view of the suction valve mounted between the first half-shell and the second half-shell.

FIG. 14 shows a preferred embodiment of a fourth fixing portion according to the present disclosure.

FIG. 15 shows the preferred embodiment of the fourth fixing portion of FIG. 14 enlarged.

FIG. 16 shows a perspective view of a preferred embodiment of a second fixing portion according to the present disclosure.

FIG. 17 shows a sectional view of the preferred embodiment of the second fixing portion of FIG. 16.

FIG. 18 shows another sectional view of the preferred embodiment of the second fixing portion of FIG. 16.

The figures are schematic in nature and serve only to understand the disclosure. The features of the different embodiments can be interchanged among each other.

### DETAILED DESCRIPTION

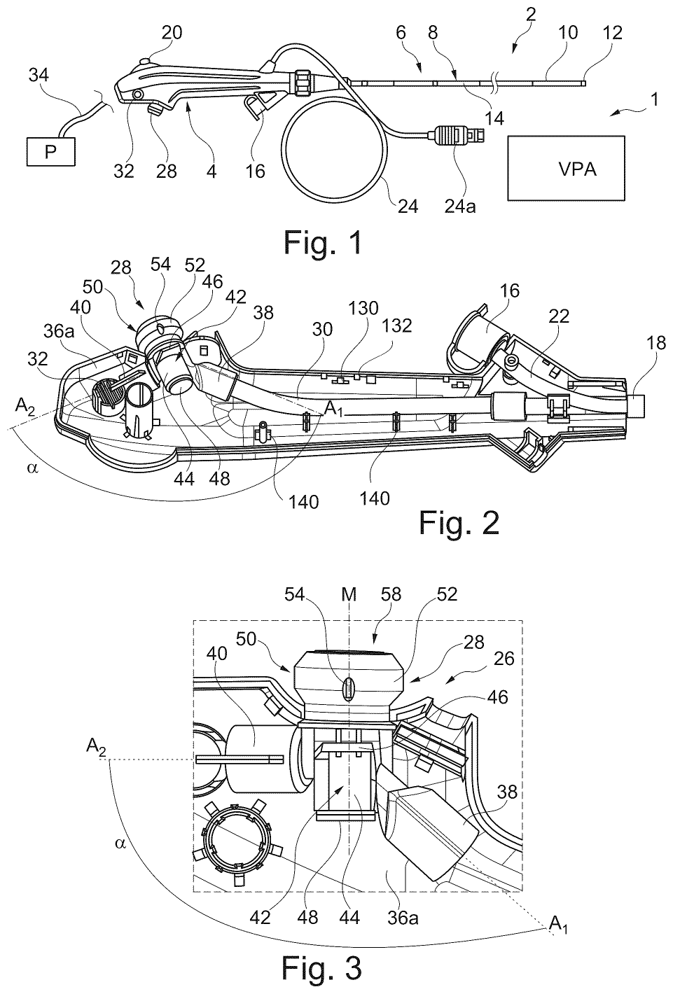

FIG. 1 shows a side view of a visualization system 1 including an endoscope 2 according to the present disclosure, which is preferably a single-use endoscope, and a video processing apparatus (VPA). The endoscope 2 comprises: a handle 4 and an insertion cord 6 extending distally from the handle 4. The insertion cord 6 is configured to be inserted into a patient's body cavity and comprises an insertion tube 8, a bending section 10 and a distal tip unit 12. The endoscope 2 further comprises a working channel 14 which extends from a biopsy port or working channel access port 16 provided at the handle 4 to the distal tip unit 12. The working channel 14 comprises a working channel tube 18 (shown in FIG. 2), which is arranged inside the handle 4, the insertion tube 8, and the bending section 10. The handle 4 comprises an operating unit 20, formed as a lever, for bending the bending section 10 of the insertion cord 6. The working channel tube 18 and the working channel access port 16 are connected via a Y-connector 22. The endoscope 2 is connectable to a video processing apparatus VPA, which is schematically illustrated via a connecting cable 24 comprising an electrical connector 24a insertable in a socket (not shown) of the video processing apparatus VPA. Alternatively the endoscope 2 may be wirelessly connectable to the video processing apparatus VPA, e.g. by a wireless HDMI connection.

When a surgical instrument or tool is inserted into the working channel 14, the surgical instrument or tool is inserted into the working channel access port 16 and guided through the Y-connector 22 into the working channel tube 18.

The endoscope 2 further comprises a suction valve 26, having a suction button 28 and configured to control a suction power through the working channel 14 of the endoscope 2. The suction valve 26 is connected to a suction channel 30 and a vacuum port 32, which protrudes from or is embedded in the handle 4 (shown in FIG. 2). The endoscope 2 is connectable to a suction pump P, which is schematically illustrated in FIG. 1, via a hose 34, which is connectable to the vacuum port 32 of the endoscope 2.

FIG. 2 shows the handle 4 in an open state. The handle 4 is formed from two half-shells 36, a first half-shell 36a and a second half-shell 36b, that form the housing of the handle 4. In FIG. 2, the second half-shell 36b is removed and elements of the endoscope 2 which are not necessary for an understanding of the present disclosure are also not shown in FIG. 2. Mechanical, optical and electronic components, which are comprised in the handle 4 in a conventional endoscope are or may be comprised in the endoscope 2 of the present disclosure.

The two half-shells, or handle housing shells, 36 are connected with snap-fit shell fasteners 130, each including a snap lug and a snap hook. The snap lug is placed on one housing shell and a corresponding snap hook is placed on the other housing shell. When the housing shells 36 are pressed together the snap hooks engage with the snap lugs to attach the housing shells to each other. The snap hooks and snap lugs are also referred to, below, as the retention hooks 72a and counterparts 72b. In the range of 6-12 snap-fit shell fasteners provide a strong and stable connection between the housing shells. In one variation, 7-9 snap-fit shell fasteners 30′ connect the two housing shells. The two half-shells 36 may also comprise press-fit component fasteners 140. A press-fit component fastener may comprise a stud or rod and a cavity. The stud can be on a handle shell and the cavity be part of a component to be attached thereto, or vice versa, preferably without adhesives. The two half-shells 36 may also comprise alignment means 132, i.e., pairs of a first alignment part and a second alignment part, may be in the range of 5-13, preferably in the range of 6-10. The combination of the snap-fit shell fasteners, press-fit component fasteners, and optionally alignment means, form a three-dimensional support network that has the effect that when the two housing shells are snapped together, they cannot be displaced relative to each other in any direction. Also, along the handle split line the connection will be firm and robust. By preventing displacement, in particular due to the use of the alignment means 132, it is also prevented that the shells will un-snap and separate. The three-dimensional network comprises a plurality of alignment means and snap-fit shell fasteners. The three-dimensional network is suitable particularly when the shells are made from relatively more flexible material, such as PP, even more particularly when a wall thickness of a shell is thin, to reduce material costs and carbon footprint. In one example, the wall thickness of more than 70% of the shell is 2.5 mm or less, preferably 2.0+/−0.2 mm. The three-dimensional support network, snap-fit shell fasteners, press-fit component fasteners, and the alignment means, are described in more detail in U.S. Patent Application No. (tbd, docket no. AMBU1270US, which claims priority from European Patent Application No. 24 1602 40.8, filed Feb. 28, 2024), titled “Handle for an endoscope,” the disclosure of which is incorporated herein by reference in its entirety.

The suction channel 30 connects the suction valve 26, which is mounted between the first half-shell 36a and the second half-shell 36b at a proximal end portion of the handle 4, to the Y-connector 22 and runs in a longitudinal direction of the handle 4, that is, essentially in a direction, which continues the insertion cord 6. The suction valve 26 comprises an inlet 38, which is configured to connect to the suction channel 30. In other words, the inlet 38 is configured as a connector, which connects to the suction channel 30. The suction channel 30 has a tubular shape and may be in the form of an elastomeric tube or a rigid pipe. The suction valve 26 further comprises an outlet 40, which is connectable to the vacuum port 32. In other words, the outlet 40 is configured as a connector, which connects to the vacuum port 32.

In the following, the external structure of the suction valve 26 is explained in more detail with reference to FIGS. 2 and 3. FIG. 3 is an enlarged view of the suction valve 26 in the assembled state, whereby the suction channel 30 and the vacuum port 32 are not shown in FIG. 3.

The suction valve 26 comprises a valve body 42, which comprises a valve cylinder 44 extending in an axial direction of the suction valve 26. The valve body 42 further comprises the inlet 38 and the outlet 40. The inlet 38 and the outlet 40 each have a substantially tubular geometry with a substantially circular cross-sectional configuration. Seen in the side view of the handle 4 in an assembled state of the suction valve in FIG. 3, the inlet 38 has a first center axis A1 and the outlet 40 has a second center axis A2. The first center axis A1 extends in a center position of the inlet 38 in a direction of extension of the inlet 38. The second center axis A2 extends in a center position of the outlet 40 in a direction of extension of the outlet 40. An angle α is enclosed between the first center axis A1 and the second center axis A2. The angle α is greater than 90° and less than 180°. Preferably, the angle is greater than 110°. Particularly preferably, the angle is about 120°. The angles and other features of the body of the suction valve are described in more detail in U.S. Patent Application No. (tbd, docket no. AMBU1263US, which claims priority from European Patent Application No. 23 195 488.4, filed Sep. 5, 2023), titled “ENDOSCOPE COMPRISING A SUCTION VALVE,” the disclosure of which is incorporated herein by reference in its entirety.

The valve body 42 is formed in one piece from a plastic material, preferably polypropylene. Further, the valve body 42 comprises fixing projections 46 formed thereon, which are configured to fix the suction valve 26 in the handle 4 between the half-shells 36a and 36b. The suction valve 26 includes two fixing projections 46 which extend oppositely radially outwardly away from the valve cylinder 44. The fixing projection 46 will be described in more detail below.

The suction valve 26 further includes an end cap 48 formed on an end portion of the valve cylinder 44 facing an interior of the handle 4, and closing the valve cylinder 44. The end cap 48 closes a substantially circular bottom opening of the valve body 42. The end cap 48 and the valve body 42 are welded together. Alternatively, it is conceivable that the end cap 48 may be fixed in place by adhesive bonding or by a latching action.

A valve spring 50 is formed on an end portion of the valve cylinder 44 opposite the end cap 48. The valve spring 50 protrudes for the most part from the housing of the handle 4 and ensures that the suction valve 26 is maintained in an initial valve closed state or is returned to the initial valve closed state without a user actuation. A bypass hole 54 is formed in a side surface 52 of the valve spring 50. The bypass hole 54 allows ambient air to be drawn through the suction valve 26 into the outlet 40 in a closed state of the suction valve, that is, in a state in which the volume flow from inlet 38 to outlet 40 is blocked.

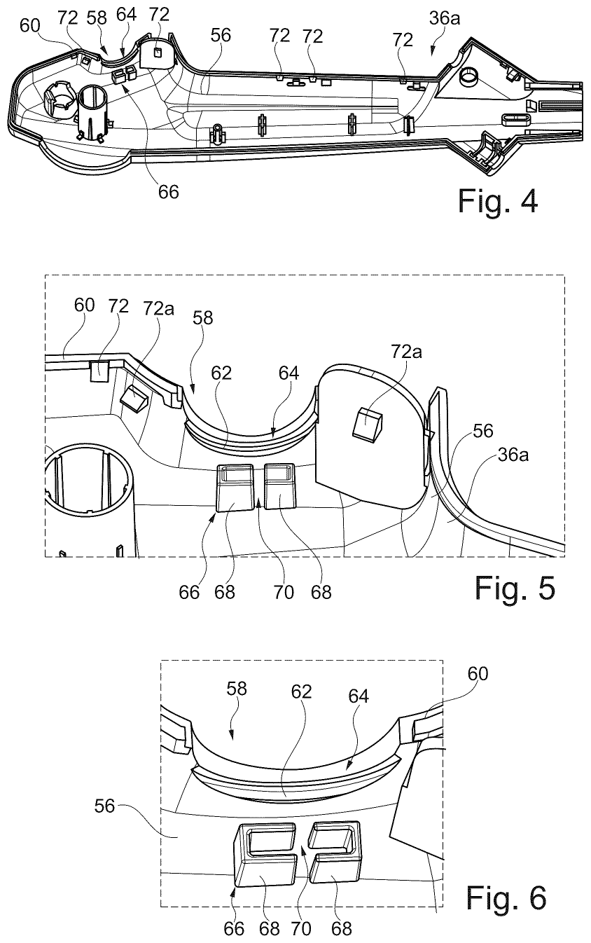

FIG. 4 shows the first half-shell 36a. FIG. 5 shows an enlarged view of the first half-shell 36a in an area of the first half-shell 36a in which the suction valve 26 is received. FIG. 6 shows a further enlarged view of receiving geometries in said area. In the following, the receiving geometries for the suction valve 26 in the first half-shell 36a are described in more detail with reference to FIGS. 4, 5 and 6.

The first half-shell 36a comprises a shell skin 56 forming an outer wall of the handle 4. A recess 58 is formed in the shell skin 56, the recess 58 being configured to allow the suction valve 26 to extend outwardly from the interior of the handle 4 through the shell skin 56 of the handle 4. The recess 58 has a shape substantially in the form of a semicircle and extends from a contact edge 60 at which the first half-shell 36a and the second half-shell 36b abut one another in an assembled state of the handle 4. The recess 58 of the first half-shell 36a is configured to form a circular opening in the shell skin 56 of the handle 4 in conjunction with the recess 58 of the second half-shell 36b when the first half-shell 36a and the second half-shell 36b are assembled (shown in FIG. 9).

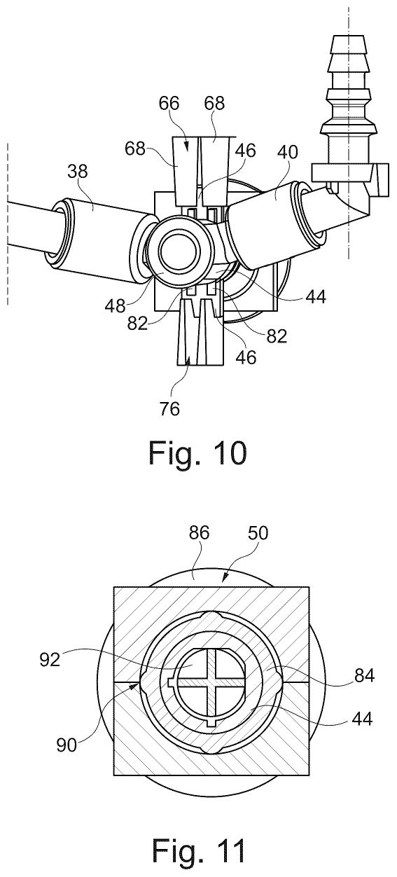

Directly adjacent to the recess 58, towards an interior space enclosed by the handle 4, a groove 62 is formed which, in conjunction with the recess 58, forms a first outer fixing portion 64 according to the preferred embodiment. The groove 62 extends into the shell skin 56, starting from the contact edge 60 in the area of the recess 58. The groove 62 may extend completely around the recess 58 or around a portion of the recess 58. The function of the first fixing portion 64 is described in more detail below with reference to FIGS. 10 to 13.

Parallel to the groove 62, offset towards the interior space of the handle 4, that is, further away from the recess 58, a second inner fixing portion 66 extends, starting from the shell skin 56, perpendicularly into the interior space. The second fixing portion 66 comprises two U-shaped brackets 68 extending away from the shell skin 56 facing each other and forming a substantially rectangular or slot-shaped receptacle 70. The rectangular receptacle 70 is oriented parallel to the groove 62 and forms a cuboid-shaped receiving space. The two U-shaped brackets 68 face each other with arm sections of the U-shape. The two U-shaped brackets 68 extend from the shell skin 56 in a tower shape. The function of the second fixing portion 66 is described in more detail below with reference to FIGS. 10 to 13.

Embodiments are also conceivable, in which the receptacle 70 slightly tapers toward the shell skin 56 as shown in FIGS. 16 to 18.

Furthermore, embodiments are conceivable which are not formed from two U-shaped brackets 68 but from a single, preferably rectangular element having the rectangular or slot-shaped receptacle 70.

The first half-shell 36a is a one-piece component injection molded from a plastic, preferably a thermoplastic. The second half-shell 36b is a one-piece component injection molded from a plastic, preferably a thermoplastic. The thermoplastic material may comprise at least 20% polypropylene. The handle, the valve body and the stem, may each comprise at least 20% polypropylene material. The polypropylene material may be bio-propylene. The walls of each of the half-shells may comprise an outer surface and an inner surface, the wall having an average wall thickness of 2.5 mm or less.

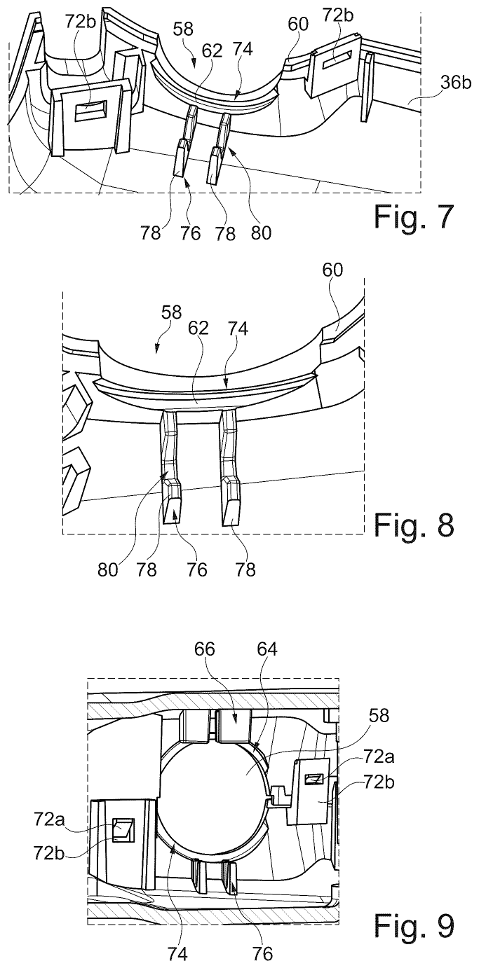

Adjacent to the recess 58, retention hooks 72a are formed on the first half-shell 36a on the shell skin 56, which are configured to latch into corresponding counterparts 72b (shown in FIG. 7) in the second half-shell 36b to connect the first half-shell 36a and the second half-shell 36b together. Such retention hooks 72a are further evenly distributed around the entire circumference of the first half-shell 36a, preferably along the contact edge 60.

FIG. 7 and FIG. 8 show the receiving geometries for the suction valve 26 in the second half-shell 36b. Since the second half-shell 36b corresponds in large parts to the first half-shell 36a, only differences between the second half-shell 36b and the first half-shell 36a will be described in the following.

Directly adjacent to the recess 58 of the second half-shell 36b, towards an interior space enclosed by the handle 4, the groove 62 is formed which, in conjunction with the recess 58, forms a third outer fixing portion 64. The groove 62 extends into the shell skin 56, starting from the contact edge 60 in the area of the recess 58.

Thus, the third fixing portion 74 of the second half-shell 36b corresponds to the first fixing portion 64 of the first half-shell 36a. In other words, the first fixing portion 64 and the third fixing portion 74 are identical in configuration. In still other words, the first fixing portion 64 and the third fixing portion 74 are symmetrical to each other with respect to a splitting layer or dividing plane of the handle 4. The splitting layer is a layer, which contains the contact edge 60.

Parallel to the groove 62, offset towards the interior space of the handle 4, that is, further away from the recess 58, a fourth inner fixing portion 76 extends, starting from the shell skin 56, perpendicularly into the interior space of the handle 4. The fourth fixing portion 76 comprises two V-shaped brackets 78 extending away from the shell skin 56 facing each other and forming a substantially V-shaped receptacle 80. The V-shaped receptacle 80 tapers from the interior space of the handle 4 to the shell skin 56. The V-shaped receptacle 80 is oriented parallel to the groove 62 and forms a three-sided straight prism-shaped receiving space. In other words, a receiving space that tapers toward the shell skin 56. The two V-shaped brackets 78 are each formed in a plate shape and parallel to each other. The two V-shaped brackets 78 extend from the shell skin 56 in a tower shape. The function of the second fixing portion 66 is described in more detail below with reference to FIGS. 10 to 13.

Furthermore, embodiments are conceivable which are not formed from two V-shaped brackets 78 but from a single, preferably rectangular element having the three-sided straight prism-shaped receiving space shaped receptacle 80.

In one embodiment, a portion of the V-shaped receptacle 80 that is formed closest to the shell skin 56 may include a parallel slot-shaped portion. In other words, the V-shaped receptacle 80 can change over its course into a regular rectangular slot shape.

Adjacent to the recess 58, the counterparts 72b are formed on the second half-shell 36b on the shell skin 56, which are configured to latch into the corresponding retention hooks 72a.

FIG. 9 shows the first half-shell 36a attached to the second half-shell 36b. The retention hooks 72a of the first half-shell 36a are mounted in the counterparts 72b of the second half-shell 36b. The recess 58 is configured as a circular opening in shell skin 56. The retention hooks 72a and the counterparts 72b form a snap-fit shell fastener 130.

The first fixing portion 64 of the first half-shell 36a is positioned opposite the third fixing portion 74 of the second half-shell 36b. The second fixing portion 66 of the first half-shell 36a is positioned opposite the fourth fixing portion 76 of the second half-shell 36b.

FIG. 10 shows the suction valve 26 mounted in the first fixing portion 64, the second fixing portion 66, the third fixing portion 74 and the fourth fixing portion 76. A visualization of the half-shells 36 has been omitted for the sake of clarity. The fixing projections 46 are formed on the suction valve 26, which extend radially outward away from the valve cylinder 44. The fixing projections 46 are plate-shaped geometries, which are formed with supporting ribs 82. The ribs 82 also extend radially away from the valve cylinder 44 and extend over portions of the fixing projection 46. Two ribs 82 are formed on each of the fixing projections 46.

In the assembled state, a first of the fixing projections 46 is accommodated in the second fixing portion 66. In other words, the first of the fixing projections 46 is inserted into the second fixing portion 66 in such a way that the two U-shaped brackets 68 circumferentially or peripherally surround the first of the fixing projections 46 and the first of the fixing projections 46 is in the cuboid-shaped receiving space.

The ribs 82 provided on the first of the fixing projections 46 form a stop. In other words, the first of the fixing projections 46 can only be inserted into the second fixing portion 66 until the ribs 82 abut the second fixing portion 66.

A second of the fixing projections 46 is inserted in the fourth fixing portion 76. In other words, the second of the fixing projections 46 is inserted into the fourth fixing portion 76 in such a way that the two V-shaped brackets 68 accommodate the second of the fixing projections.

Further, the suction valve 26 is mounted to the first fixing portion 64 and the third fixing portion 74. The valve spring 50 of the suction valve 26 is connected to the housing of the handle 4 via the first fixing portion 64 and the third fixing portion 74.

The fixation of the valve spring 50 is shown in FIG. 11 and will be explained in more detail below.

The valve spring 50 comprises a collar or flange 84 and a valve spring main body 86. The collar 84 is fixed by the first fixing portion 64 and the third fixing portion 74. The collar 84 and the valve spring main body 86 are connected to each other by a neck 88 (shown in FIG. 13). The first fixing portion 64 and the third fixing portion 74 surround the valve spring 50 in the area of the neck 88 in such a way that they fully surround the valve spring 50 in the area of the neck 88. The collar 84 is thereby positioned in the groove 62. The collar 84 comprises four tolerance absorbing bumps 90, which are each offset by 90° around the circumference of the collar 84.

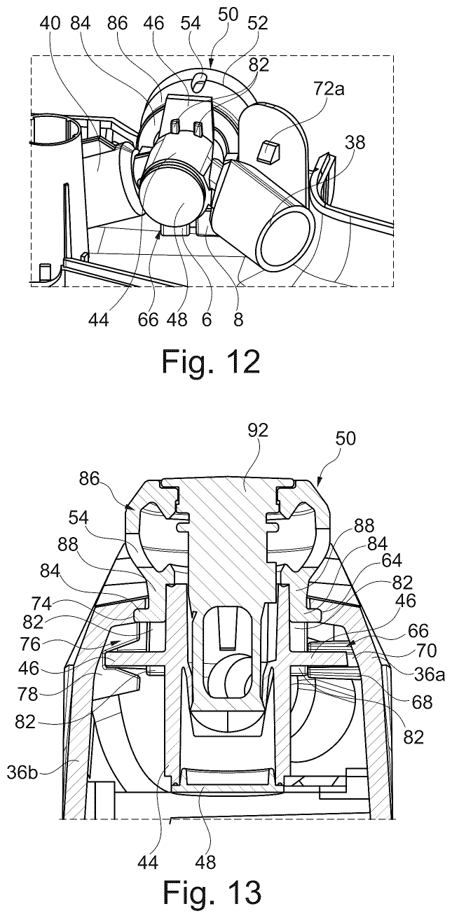

In the following, the assembly of the suction valve 26 in the handle 4 is explained in more detail with reference to FIGS. 12 and 13.

In a first step, shown in FIG. 12, the suction valve 26 is positioned in the first half-shell 36a. For this purpose, the first of the fixing projections 46 is inserted into the second fixing portion 66 and the neck 88 of the valve spring 50 is positioned in the first fixing portion 64. The first of the fixing projections 46 is slid between the two U-shaped brackets 68 until the ribs 82 rest against the U-shaped brackets 68. Further, the collar 84 is positioned in the groove 62.

In a second step shown in FIG. 13, the first half-shell 36a and the second half-shell 36b are put together. Therefore, the second of the fixing projections 46 is inserted into the fourth fixing portion 76. The V-shape of the fourth fixing portion 76 makes insertion of the second of the fixing projections 46 into the fourth fixing portion 76 much easier. Furthermore, the neck 88 of the valve spring 50 is positioned in the third fixing portion 74. Therefore, the collar 84 is positioned in the groove 62. Subsequently, the handle 4 is assembled from the first half-shell 36a and the second half-shell 36b by snapping the retention hooks 72a into the counterparts 72b. Other electrical and mechanical components and assemblies may be positioned in the handle 4 prior to assembling the first half-shell 36a and the second half-shell 36b.

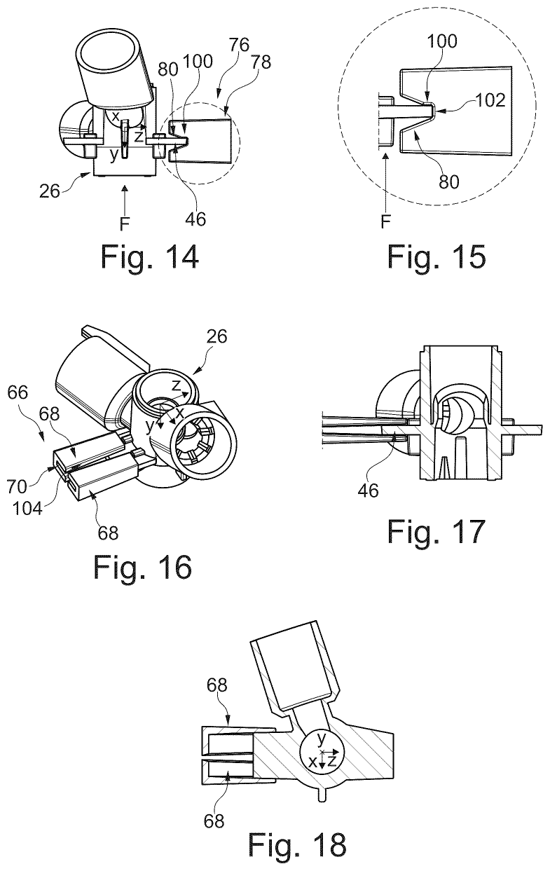

FIG. 14 shows a preferred embodiment of a fourth fixing portion 76. Here, the fourth fixing portion 76 also comprises two V-shaped brackets 78 (only one shown) as second tower geometries extending away from the shell skin 56 facing each other and forming a substantially V-shaped receptacle 80. The V-shaped receptacle 80 is configured to guide a fixing projection 46 of the suction valve 26 inside. A xyz-coordinate system is shown for reference.

FIG. 15 shows the fourth fixing portion 76 of FIG. 14 enlarged. The V-shaped receptacle 80 comprises a force-taking surface portion 100. A force F is shown that results from pressing or pushing the suction button 28. The force-taking surface portion 100 is an inner surface portion of the V-shaped receptacle that is perpendicular to the force F. Thereby, the force F that is (partially) transferred to the projection portion 46 is taken from the force-taking surface portion 100 providing stability. The force-taking surface portion 100 taking the brunt of the force to the V-shaped receptacle 80) prevents the suction valve 26 from moving in y-direction and rotating around the x-axis due to the force F.

Further, the V-shaped receptacle 80 comprises a flat or planar inner end surface portion 102. The end surface portion is the inner surface closest to the shell skin 56 of the second half-shell 36b. The end surface portion 102 prevents the suction valve 26 from moving (further) in z-direction.

FIG. 16 shows a preferred embodiment of a second fixing portion 66 according to the disclosure. In this embodiment, the second fixing portion 66 also comprises two U-shaped brackets 68 facing each other. The two U-shaped brackets 68 are first tower geometries protruding from the first half-shell 36a in the direction of the interior of the handle 4 and forming a split slot 104 in between them. The receptacle 70 of the second fixing portion 66 is slot-shaped. Here, the slot-shaped receptacle 70 is configured for a press-fit with the fixing projection 46 and/or they form a press-fit. A xyz-coordinate system is shown for reference.

FIG. 17 shows a sectional view of the embodiment of FIG. 16 along a cutting plane parallel to the yz-plane. One fixing projection 46 of the suction valve 26 is inserted in the receptacle 70 of the second fixing portion 66. The receptacle 70 slightly tapers in the yz-plane continuously and linearly. This enables a press-fit between the slot-shaped receptacle 70 and the fixing portion 66. The receptacle 70 has at least one angled interface also serving as an inlet chamfer.

FIG. 18 shows another sectional view of the embodiment of FIG. 16, in this case along a cutting plane parallel to the yz-plane. As shown here, the receptacle 70 also tapers continuously and linearly in this plane as a second dimension. In other words, in this preferred embodiment of FIGS. 16 to 18, the receptacle has a rectangular cross-section tapering, i.e. becoming narrower in width and height further inside the receptacle.

Comparing the preferred embodiments of the V-shaped receptacle 80 of FIG. 14 and the slot-shaped receptacle 70 of FIG. 16, it is apparent that in this case the second, inner fixing portion 66 with the slot-shaped receptacle 70 suppresses more degrees of freedom of the suction valve 26 than the fourth, inner fixing portion 76 with V-shaped receptacle 80.

Due to the shape of the slot-shaped receptacle 70 the second, inner fixing portion 66 is configured or shaped to suppress translational movements of the suction valve 26 relative to the housing of the endoscope except in direction of the second half-shell 36b for disassembly when the fixing projection 46 is inserted in an assembled state of the endoscope, wherein the fourth, inner fixing portion 76 with the V-shaped receptacle 80 is configured/shaped to prevent this movement and due its shape is configured to guide the suction valve in that direction inwards during assembly, making the assembly easier.

The slot-shaped receptacle 70 of the second, inner fixing portion 66 is configured to suppress by its shape translational movements (inward and outward of the housing) and (arbitrary) rotational movements of the suction valve 26 relative to the housing of the endoscope, except for translational movements in direction of the second half-shell 36b, so that it is possible to take the fixing projection 46 out of the receptacle during disassembly of the endoscope 2. It provides, with its shape due to the angled interfaces, some small rotational looseness or clearance for the fixing projection 46 making the assembly easier. The rotational looseness or clearance may be suppressed by the press-fit.

The following items are further variations and examples of the embodiments described with reference to the figures.

1. An endoscope (2) comprising: a proximal handle or interface (4); an insertion cord (6) extending from the handle or interface (4) and configured to be inserted into a patient's body cavity; a working channel (14) configured to let fluid and mucus sucked from the patient's body cavity flow therethrough; the handle or interface (4) comprising: a housing; and a suction valve (26) configured to control a suction through the working channel (14) and having a valve closed state and a valve open state; the housing comprising a first half-shell (36a) and a second half-shell (36b); the suction valve (26) comprising: a valve body (42) having: an inlet connected (38) to the working channel (14); an outlet (40) provided and configured to be connected to a suction device (P); and a stem (92) movable axially in the valve body (42) between at least a first position, in which the suction valve (26) is in the valve closed state, and a second position, in which the suction valve is in the valve open state; wherein the suction valve (26) is mounted between the first half-shell (36a) and the second half-shell (36b); and wherein the first half-shell (36a) comprises a first, outer fixing portion (64) and a second, inner fixing (66) portion, wherein the first, outer fixing portion (64) is configured to receive a first portion of the suction valve (26) and wherein the second, inner fixing portion (66) is configured to receive a second portion of the suction valve (26), the second half-shell (36b) comprises a third, outer fixing portion (74) and a fourth, inner fixing portion (76), wherein the third, outer fixing portion (74) is configured to receive the first portion of the suction valve (26), and wherein the fourth, inner fixing portion (76) is configured to receive the second portion of the suction valve (26), wherein the second, inner fixing portion (66) of the first half shell (36a) and the fourth inner fixing portion (76) of the second half shell (36b) have a different geometry, and wherein the second, inner fixing portion (66) is configured to suppress at least one degree of freedom of the suction valve (26) and the fourth, inner fixing portion (76) is configured to suppress a remaining degree or remaining degrees of freedom of the suction valve (26), for fixing the suction valve in its position and orientation in the housing.

2. The endoscope (2) according to item 1, wherein the second, inner fixing portion (66) is configured to suppress more degrees of freedom of the suction valve (26) than the fourth, inner fixing portion (76).

3. The endoscope (2) according to item 1 or 2, wherein the second, inner fixing portion (66) is configured to suppress by its shape translational movements and arbitrary rotational movements of the suction valve (26) relative to the housing of the handle or interface (4) except for a translational movement in direction of the second half-shell (36b), and wherein the fourth, inner fixing portion (76) is configured to suppress the translational movement in direction of the second half-shell (36b).

4. The endoscope (2) according to any of items 1 to 3, wherein the second, inner fixing portion (66) of the first half-shell (36a) comprises a slot-shaped receptacle (70) configured to hold a fixing projection (46) of the suction valve (26).

5. The endoscope (2) according to item 4, wherein the slot-shaped receptacle (70) of the second, inner fixing portion (66) tapers and forms a press-fit with the fixing projection (46) of the suction valve (26).

6. The endoscope (2) according to item 4 or 5, wherein the second, inner fixing portion (66) comprises two U-shaped brackets (68) that form the slot-shaped receptacle (70) and a split slot (104) in between them.

7. The endoscope (2) according to any of items 1 to 6, wherein the fourth, inner fixing portion (76) of the second half-shell (36b) comprises a V-shaped receptacle (80) configured to guide in a fixing projection (46) of the suction valve (26) during assembly.

8. The endoscope (2) according to item 7, wherein the V-shaped receptacle (80) comprises a flat force-taking surface portion (100) perpendicular to a direction in which a button (28) of the suction valve (26) is pushable to move the stem (92), so that when the button (28) is pushed, a movement of the suction valve (26) in that direction relative to the housing is suppressed by the flat force-taking surface portion (100).

9. The endoscope (2) according to any of items 1 to 8, wherein the second, inner fixing portion (66) of the first half-shell (36a) comprises a slot (70), preferably of constant width, and the fourth, inner fixing portion (76) of the second half-shell (36b) comprises a V-shaped slot (80).

10. The endoscope (2) according to any of items 1 to 9, wherein the valve body includes a valve cylinder; wherein the suction valve further comprises an inlet connected to the working channel, the inlet also connected to and extending away from the valve cylinder; an outlet adapted to be connected to a suction device, the outlet also connected to and extending away from the valve cylinder; a stem comprising a curved flow guide and being movable axially in the valve cylinder between at least a first position, in which the suction valve is in a valve closed state, and a second position, in which the suction valve is in a valve open state, the stem being configured to guide the fluid from the inlet to the outlet in the valve open state, wherein the inlet, the outlet and the curved flow guide are configured to provide a curved sideways flow path for the fluid through the suction valve in the valve open state.

11. The endoscope (2) according to items 10, wherein the curved sideways flow path is provided by the curved flow guide of the stem in combination with an enclosed angle, between a first orientation or center axis of the inlet and a second orientation or center axis of the outlet, being larger than 90° and smaller than 180° seen in a side view of the handle or interface in an assembled state.

12. The endoscope of item 10, wherein the curved sideways flow path is provided by the curved flow guide of the stem in combination with an enclosed angle, between a first orientation or center axis of the inlet and a second orientation or center axis of the outlet, being larger than 90° and smaller than 180° seen in a side view of the handle or interface in an assembled state, wherein the stem comprises a sealing edge, and wherein the valve body comprises a valve seat configured to make circumferential contact with the sealing edge of the stem in the valve closed state.

13. The endoscope of item 10, wherein the stem comprises a sealing edge, and wherein the valve body comprises a valve seat configured to make circumferential contact with the sealing edge of the stem in the valve closed state.

14. The endoscope (2) according to any of items 1 to 13, wherein the proximal handle (4) comprises polypropylene, preferably consists essentially of polypropylene, and/or comprises at least 70% polypropylene. In one example, the two housing shells of the housing of the handle have an average wall thickness of 2.5 mm or less, preferably 2.0+/−0.2 mm. In one example, more than 70% of the wall of each housing shell has a wall thickness of 2.5 mm or less, preferably 2.0+/−0.2 mm.

15. The endoscope (2) according any of items 1 to 13, wherein the proximal handle (4) comprises polypropylene, preferably consists essentially of polypropylene, and/or comprises at least 70% polypropylene, said propylene comprising at least 20% bio-polypropylene.

16. The endoscope (2) according to items 14 or 15, wherein the proximal handle (4) comprises two housing shells assembled with snap-fit shell fasteners without adhesives.

17. The endoscope (2) according to item 16, wherein the proximal handle (4) comprises press-fit component fasteners securing components of the endoscope within the shell without adhesives. A three-dimensional support network comprising snap-fit shell fasteners and press-fit component fasteners, the snap-fit shell fasteners and the press-fit component fasteners, respective alignment means for aligning the edges of the housing shells upon each other, are described in detail in U.S. Patent Application No. tbd (tbd, docket no. AMBU1270US, which claims priority from European Patent Application No. 24 1602 40.8), titled “Handle for an endoscope,” the disclosure of which is incorporated herein by reference in its entirety.

18. A method of assembling an endoscope (2) according to any of items 1 to 9, comprising: sliding the valve spring (50) onto the valve body (42) of the suction valve (26) and latching the valve spring (50) to the stem (92) of the suction valve (26); positioning the suction valve (26) in the first half-shell (36a) of the housing of an handle or interface (4) by inserting the second portion of the suction valve (26) in the second, inner fixing portion (66) of the first half-shell (36a) of the housing; inserting the first portion of the suction valve (26) in the first, outer fixing portion (64) of the first half-shell (36a); positioning the second half-shell (36b) of the housing on the first half-shell (36a) of the housing; inserting the second portion of the suction valve (26) in the fourth, inner fixing portion (76) of the second half-shell (36b) of the housing; inserting the first portion of the suction valve (26) in the third, outer fixing portion (74) of the second half-shell (36b); and closing the half-shells (36a; 36b).

19. A system comprising an endoscope (2) according to one of items 1 to 17 and a video processing apparatus (VPA).

### LIST OF REFERENCE NUMBERS

2 endoscope
4 handle
6 insertion cord
8 insertion tube
10 bending section
12 distal tip unit
14 working channel
16 working channel access port
18 working channel tube
20 operating unit
22 Y-connector
24 connecting cable
24a electrical connector
26 suction valve
28 suction button
30 suction channel
32 vacuum port
34 hose
36 half-shell
36a first half-shell
36b second half-shell
38 inlet
40 outlet
42 valve body
44 valve cylinder
46 fixing projection
48 end cap
50 valve spring
52 side surface
54 bypass hole
56 shell skin
58 recess
60 contact edge
62 groove
64 first fixing portion
66 second fixing portion
68 U-shaped bracket
70 slot-shaped receptacle
72a retention hook
72b counterpart
74 third fixing portion
76 fourth fixing portion
78 V-shaped bracket
80 V-shaped receptacle
82 rib
84 collar/flange
86 spring main body
88 neck
90 bump
92 stem
A1 first center axis
A2 second center axis
α angle
P suction pump
VPA video processing apparatus
100 force-taking surface portion
102 end surface portion
104 split slot

1. An endoscope comprising:
a handle including a housing and a suction valve, the housing comprising a first half-shell and a second half-shell, the first half-shell including an outer fixing portion and an inner fixing portion, the second half-shell including an outer fixing portion and an inner fixing portion having a different geometry than the inner fixing portion of the first half-shell, the suction valve being mounted between the first half-shell and the second half-shell and comprising a valve body, a stem, a first portion and an second portion, the valve body having an inlet and an outlet, and the stem being movable axially in the valve body to open and close the suction valve to control a suction through the suction valve;
an insertion cord extending from the handle and configured to be inserted into a patient; and
a working channel extending through the insertion cord and being connected to the inlet of the suction valve and configured to suction fluids from the patient;
wherein the outer fixing portion and the inner fixing portion of the first half-shell receive, respectively, the first portion and the second portion of the suction valve,
wherein the outer fixing portion and the inner fixing portion of the second half-shell receive, respectively, the first portion and the second portion of the suction valve, and
wherein the inner fixing portion of the first half-shell is configured to suppress at least one degree of freedom of the suction valve and the inner fixing portion of the second half-shell is configured to suppress a remaining degree or remaining degrees of freedom of the suction valve, the inner fixing portion of the first half-shell and the inner fixing portion of the second half-shell fixing a position and an orientation of the suction valve in the housing.

2. The endoscope of claim 1, wherein the inner fixing portion of the first half-shell is configured to suppress more degrees of freedom of the suction valve than the inner fixing portion of the second half-shell.

3. The endoscope of claim 1, wherein the handle, the valve body and the stem, are each comprised of at least 20% polypropylene material.

4. The endoscope of claim 1, wherein the first half-shell comprises a wall having an outer surface and an inner surface, the wall having an average wall thickness of 2.5 mm or less, and wherein the handle comprises at least 20% bio-polypropylene material.

5. The endoscope of claim 1, wherein the inner fixing portion of the first half-shell suppresses translational movement of the suction valve relative to the housing of the handle.

6. The endoscope of claim 1, wherein the inner fixing portion of the first half-shell suppresses translational movement of the suction valve relative to the housing of the handle along an axial direction of the stem but allows a translational movement of the suction valve in a direction toward the second half-shell, and wherein the inner fixing portion of the second half-shell is configured to suppress the translational movement in the direction toward the second half-shell.

7. The endoscope of claim 1, wherein the suction valve comprises a fixing projection, wherein the inner fixing portion of the first half-shell comprises a slot-shaped receptacle holding the fixing projection of the suction valve, and wherein the slot-shaped receptacle tapers and forms a press-fit with the fixing projection of the suction valve.

8. The endoscope of claim 1, wherein the suction valve comprises a fixing projection, and wherein the inner fixing portion of the first half-shell comprises a slot-shaped receptacle holding the fixing projection of the suction valve.

9. The endoscope of claim 8, wherein the slot-shaped receptacle tapers and forms a press-fit with the fixing projection of the suction valve.

10. The endoscope of claim 8, wherein the inner fixing portion of the first half-shell comprises two U-shaped brackets and a split slot in between them, the U-shaped brackets and the split slot forming the slot-shaped receptacle.

11. The endoscope of claim 1, wherein the suction valve comprises a fixing projection, wherein the inner fixing portion of the second half-shell comprises a V-shaped receptacle configured to guide in the fixing projection of the suction valve during assembly.

12. The endoscope of claim 11, wherein the suction valve comprises a button pushable to translate the stem axially, wherein the V-shaped receptacle comprises a flat force-taking surface portion perpendicular to a direction in which the button of the suction valve is pushable to translate the stem.

13. The endoscope of claim 12, wherein the flat force-taking surface portion suppresses movement of the suction valve, relative to the housing, parallel to the axial direction of the stem.

14. The endoscope of claim 1, wherein the suction valve comprises a fixing projection, wherein the inner fixing portion of the second half-shell comprises a V-shaped receptacle configured to guide in the fixing projection of the suction valve during assembly, wherein the suction valve comprises a button pushable to translate the stem axially, wherein the V-shaped receptacle comprises a flat force-taking surface portion perpendicular to a direction in which the button of the suction valve is pushable to translate the stem, and wherein the flat force-taking surface portion suppresses movement of the suction valve, relative to the housing, parallel to the axial direction of the stem.

15. The endoscope of claim 1, wherein the inner fixing portion of the first half-shell comprises a constant width slot, and wherein the inner fixing portion of the second half-shell comprises comprises a V-shaped slot.

16. The endoscope of claim 1, wherein the valve body and the stem are each comprised in one piece of a thermoplastic material comprising at least 70% polypropylene.

17. The endoscope of claim 1, wherein the handle, the valve body and the stem, are each comprised of at least 70% polypropylene material.

18. The endoscope of claim 17, wherein at least 20% of the polypropylene material is bio-propylene.

19. The endoscope of claim 18, wherein the first half-shell comprises a wall having an outer surface and an inner surface, the wall having an average wall thickness of 2.5 mm or less.

20. A system comprising the endoscope of claim 1 and a video processing apparatus.
